(19) United States
(12) Patent Application Publication
(10) Pub. No.: US 2025/0340808 A1
(43) Pub. Date: Nov. 6, 2025

(54) CELL CULTURE DEVICE

(71) Applicant: REVIVOCELL LIMITED, Warrington (GB)

(72) Inventor: Valon LLABJANI, Warrington (GB)

(21) Appl. No.: 18/864,647

(22) PCT Filed: May 10, 2023
(86) PCT No.: PCT/GB2023/051224

§ 371 Date: Nov. 11, 2024

(30) Foreign Application Priority Data
May 10, 2022 (GB) 2206803.5

(51) Int. Cl. C12M 1/32; C12M 1/00; C12M 1/12; C12M 3/00

(52) U.S. Cl. CPC C12M 23/12; C12M 23/44; C12M 23/50; C12M 25/02

(57) ABSTRACT

A cell culture system comprising a receptacle having a cell growth area and a stacking formation comprising a recess or protrusion configured to abut an adjacent receptacle in use to limit relative movement therebetween in at least one direction. The stacking formation is configured to space adjacent receptacles and comprises at least one gap therein, such that the space defined between the receptacle is in fluid communication with an environment external to the receptacle.

The present invention relates to a cell culture device, particularly but not limited to, a cell culture device for cell culture of different cell types.

### BACKGROUND OF THE INVENTION

A prior art cell culture device is shown in WO2017134464. The device comprises a tray 2 having a plurality of dividers 4 to provide respective compartments. A plurality of cell growth blocks 3 are provided in the compartments. The cell growth blocks are positioned in the tray such that the sides of the blocks are touching one another. If different types of cells are grown in each cell growth block, the close proximity enables transfer of cell growth signals between the different types of cells, but the selectively permeable walls prevent the cells from migrating into other blocks.

The inventor has found numerous problems with the prior art device. Whilst the system may be used to grow different cell types, the cell growth blocks are spaced apart from one another. Thus, the interactions between different cell types which are adjacent/connected in vivo may not be accurately represented by the system. Additionally, separation of the blocks results in different cells not being grown/connected together. Thus, the system is not suitable for simultaneously culturing multiple different cell types, for example, in order to replicate conditions in vivo.

### STATEMENT OF INVENTION

According to a first aspect of the invention, there is provided a cell culture system comprising:
a receptacle having a cell growth area;
a stacking formation comprising a recess or protrusion configured to abut an adjacent receptacle in use to limit relative movement therebetween in at least one direction;
the stacking formation configured to space adjacent receptacles and comprising at least one gap therein, such that the space defined between the receptacle is in fluid communication with an environment external to the receptacle.

The receptacle may comprise a plate or disc. A plurality of receptacles may be stacked to form a stack or cell block. A cell block may comprise from 2 to 20 receptacles, from 2 to 10 receptacles, from 2 to 6 receptacles, or four receptacles. The receptacles may allow fluid to flow therethrough such that the adjacent receptacles of a cell block are in direct fluid communication.

The stacking formation may comprise a first protrusion on a first side of the receptacle and a second protrusion on a second side of the receptacle.

The first protrusion is configured to be received within the second protrusion when stacked with an adjacent receptacle. The first protrusion may be provided radially inward relative to the second protrusion.

The second protrusion may comprise a ramped or curved portion configured to space opposing faces of the first and second in use. The ramped/curved portion may be provided at the interface between the receptacle and the protrusion.

Both the first protrusion and the second protrusion may comprise gaps therein.

The stacking formation may comprise a plurality of gaps, such that fluid may flow between the receptacles in two non-parallel directions. The fluid may flow in a radial direction. The fluid may flow in a circumferential direction. The gaps may be provided on non-parallel sides of the receptacle. The gaps may face different directions.

The stacking formation comprises one or more raised lips or flanges. The raised lip may be provided adjacent a side of the receptacle.

The gap comprises a raised protrusion, the protrusion extending away from the receptacle to a lesser extent than the stacking formation such that gap comprises a constriction or narrowing.

The stacking formation may collectively define a circular or arcuate shape.

The stacking formation may provide loose connection of the receptacles.

The stacking formation may prevent relative movement in two or more directions. The directions may be perpendicular. The directions may be about the plane of the receptable (eg horizontal direction).

The spacing between the receptacles at the gap may less than or equal to 5 mm; preferably, less than or equal 3 mm, or than or equal to 1 mm, and may be greater than or equal to 0.1 mm, 0.3 mm, 0.5 mm or 0.7 mm.

The receptacle may comprise an indent or recess on one or more side to allow handling thereof by a tool in use (eg tweezers). The indent/recess may be provided on a lateral side.

The receptacle may be received with a container configured to fluidly isolate the receptacle from the environment or a further receptacle.

The container may comprise a porous divider configured to separate at least two of the receptacles in use. The porous divider may prevent the passage of cells and particulate material but permit the passage of liquid or dissolved substances. The divider may be selectively permeable and in particular may permit the passage of some molecular species and prevent the passage of others. In particular, the divider may replicate the properties of a biological barrier such as the blood brain barrier, the GI tract, or blood vessels such as capillaries. The divider may define compartments in the container.

The porous divider may be removably received within the container. The container and/or the divider may comprise locating formations, such as corresponding recesses and protrusions, configured to prevent relative movement therebetween in at least one direction. The porous divider may be received within a supporting frame. The supporting frame may comprise a recess or groove.

The container may comprise a locating or stacking formation configured to engage the receptacle to prevent relative movement therebetween in at least one direction. The stacking formation may be shaped to receive the underside of the receptacle (ie is shaped to receive the stacking formation thereon).

The container may comprise one or more air filters. The system may comprise one or more sensors. The sensor may sense any one or more of the conditions within the container and in particular may be a biomass sensor, a pH sensor, a temperature sensor, an oxygen sensor or a carbon dioxide sensor.

The system may comprise a further container configured to receive a plurality of the containers. The further container may comprise a locating or stacking formation to prevent relative movement between the container and the further container in at least one direction. The locating or stacking formation may comprise corresponding recesses and/or protrusions, such as an elongate protrusion or ridge. The container or further container may comprise a tray or the like.

The cell blocks may comprise multiple types of receptacles. One or more receptacles in the cell block may comprise a barrier. The barrier may be configured such that the cells in the receptacle are in communication with the adjacent receptacle only through the cell growth area. The cell blocks may comprise more than one receptacle that comprise a barrier adjacent to one another.

The cell growth area may be selectively permeable and in particular may permit the passage of some molecular species and prevent the passage of others. In particular, the divider may replicate the properties of a biological barrier such as the blood brain barrier, the GI tract, or blood vessels such as capillaries.

The cell growth area may allow fluid to flow though the receptacle, such that adjacent receptacles are fluidly connected via the cell growth area in use. The cell growth area may be perforated or porous. The cell growth area may comprise a membrane. The cell growth area may be transparent or translucent.

The cell growth area may comprise a recess or well that is formed in the body of the receptacle.

The cell growth area may comprise a cell culture scaffold. The cell culture scaffold may be a 2D cell culture scaffold or a 3D cell culture scaffold.

The scaffold may be composed of natural or synthetic polymer, or hybrids of natural and synthetic polymers to create three-dimensional in vitro microenvironments to mimic the extracellular matrix (ECM) of native cells and tissues.

The scaffold material may be any suitable material, for example polymers (including hydrogels), tissue constructs, metals, glasses or ceramics. The scaffolds may be formed of synthetic or natural substances. The scaffolds, either individually or in combination, are incorporated into cell growth blocks to mimic the extracellular matrix (ECM) of natural living cells in laboratory conditions.

The scaffold may be a polymer scaffold and in particular may be a hydrogel scaffold. By hydrogel is meant a polymeric gel in which the liquid component is water.

The scaffolds may comprise one or more natural polymers, for example proteins (such as collagen, fibrin, alginate, gelatine, silk and/or genetically engineered proteins), polysaccharides (such as agarose, carboxymethylcellulose, hyaluronic acid and/or chitosan), DNA, live cells and tissue constructs or any combination of the above.

The mechanical strength of the scaffold can be controlled to maintain the properties of the scaffold such as its strength, longevity, stiffness, roughness, viscoelasticity and/or porosity.

The scaffold stiffness may be modified to replicate differing in vivo cell locations. Different stiffness or roughness is required to culture different cell types (for example a soft scaffold may be required for lung cells, but a hard scaffold may be required for bone cells). Techniques to make hydrogels with specific shear moduli (measured in kPa) and/or specific pore sizes are known (and published). For example, polyacrylamide stiffness is controlled by the relative concentration of acrylamide monomer and its cross-linker. Different types of tissue have different relative stiffness, and the scaffold may be engineered to have a stiffness to match.

Liver, mammary, brain, bone marrow and lung cells generally have a stiffness in the range 100 Pa to 2 kPa. A suitable polyacrylamide hydrogel may be produced using 3% acrylamide (v/v) and 0.06% bis-acrylamide (v/v), to give a hydrogel with Young's shear modulus of around 500 Pa.

Skin, spleen and kidney cells generally have a stiffness in the range 3 kPa to 8 kPa. A suitable polyacrylamide hydrogel may be produced using 5% acrylamide (v/v) and 0.15% bis-acrylamide (v/v), to give a hydrogel with Young's shear modulus of around 5 kPa.

Cardiac, myoblast, arterial, muscle and skeletal cells generally have a stiffness in the range 10 kPa to 20 kPa. A suitable polyacrylamide hydrogel may be produced using 10% acrylamide (v/v) and 0.1% bis-acrylamide (v/v), to give a hydrogel with Young's shear modulus of around 10 kPa.

Pre-calcified bone cells generally have a stiffness in the range 25 kPa to 40 kPa. A suitable polyacrylamide hydrogel may be produced using 10% acrylamide (v/v) and 0.3% bis-acrylamide (v/v), to give a hydrogel with Young's shear modulus of around 35 kPa.

The scaffold may be porous or non-porous. If the scaffold is porous, the pore distribution, pore structure and pore size can be controlled.

If the scaffold is porous, the pores may have a specific pore size (nm, μm or mm) to allow the movement of cells, cell signalling molecules, nutrients or test substances either through the matrix, surface or at specified locations or to remove waste substances.

Thus the scaffold may be selectively permeable to control the diffusion of specific test substances, cell signalling molecules, nutrients and other test agents, for example nanoparticles, viruses, or bacteria, through the scaffold and/or cell growth block, or from one block to another.

The porosity of the scaffold can be modified such that the diffusion of certain substances and their rate of diffusion can be selected. This could influence cell division, cell growth, cell death and other phenotypic changes designated as physiological, pharmacological or toxicological.

The scaffold material may allow the controlled diffusion, through its modified porosity, of agents (pharmaceuticals, toxins, agents, physical entities including nanoparticles, cell factors) towards a cell population growing on the outer surface of the scaffold. This replicates more faithfully exposure settings that occur in vivo within an in vitro setting-situations this could replicate include the diffusion of inhaled entities through lung cells into the systemic circulation, diffusion across the blood brain barrier, diffusion across the GI tract, diffusion across blood vessels such as capillaries into surrounding cells, and diffusion across all barriers (physiological and non-physiological) to influence adjacent cell populations.

The scaffold may permit diffusion of substances such as nutrients or drugs.

The scaffold may be manufactured with a pore size that provides a desired diffusion rate for a particular substance, or particular substances. Alternatively, the scaffold may be manufactured such that diffusion rates for the relevant substance(s) are known. For many diffusive materials, reference tables of a wide range of cation diffusion rates and anion diffusion rates are available. Alternatively, diffusion rates may be determined using known diffusion rate test equipment.

The scaffold may have varied pore sizes selected to enhance the growth of different specific cell types. For example, for fibroblasts and epithelial cells, pore size might range from 5μm to 100 μm, for endothelial cells about 25 μm, and for vascular smooth muscle cells, 63-100 μm. For tissue regeneration a minimum pore size of 100 μm can simulate mitigation conditions but the pore size is preferably 300 μm to improve bone formation and develop a network of capillaries.

The surface area of the scaffold may be modified and/or activated by incorporation of active functional groups (for example peptides) to increase support for cell growth and for mitigating scaffold degradation. The polarity of the scaffold may be modified to increase cell adhesion and the spreading of living cells, for example by adding proteins, changing the charge of the surface groups, or by the addition of peptides.

Cell adhesion may be increased by inserting structural motifs within the scaffold. For example, a negative surface charge can increase cell attachment.

The scaffold may be supported by other materials to give a specific shape (eg square) or to maintain its structure. The supporting material for the scaffold block may be any suitable material, for example, plastic, metal, ceramic or glass.

Antibodies, scaffolds or other structures (including nanostructures) could be adsorbed onto the surface of these supporting materials to selectively adhere or manipulate specific cell types for selective clonal expansion or isolation from a particular cell population.

Other proteins such as plasma proteins (eg albumin) may be contained within a scaffold; these are commonly found in blood and influence the kinetics (absorption, metabolism and excretion) of pharmaceuticals and toxic agents.

The scaffold may be formed with an outer layer which may be advantageously modified to improve cell attachment and better support cell growth and differentiation. Such a layer may comprise hydrogel. In particular, a layer of hydrogel may include an outer surface coated with an Extra-Cellular Matrix (“ECM”).

A hydrogel outer layer of the scaffold may be formed to match one or more properties of the cells and/or tissue desired to grow there. Such properties may include rigidity, stiffness and/or other native properties of different body tissues.

According to a second aspect of the invention, there is provided a cell culture method comprising:
implanting cells on/within a receptacle according to the first aspect of this invention;
implanting further cells on/within a further receptacle according to the first aspect of this invention;
stacking the receptacles such that they are in fluid communication; and
providing a growth medium to the receptacles to promote growth of the cells.

The method may further comprise implanting further cells on three, four, five, six, seven, eight or more receptacles according to the first aspect of this invention and stacking those receptacles such that they are in fluid communication.

The cells may comprise cells of a different type. The cells may comprise one or more component cells of a human or animal organ. The cells may comprise one or component cells of a liver, kidney, skin, heart, mammary tissue, brain, bone marrow, lung, or any other organ or structure of the body. The cell types may be selected such that the stack of receptacles replicates the structure of an organ or biological system.

The method may further comprise removing, adding or replacing one or more receptacles from the stack and/or rearranging the receptacles in the stack.

According to a further aspect, there is provided: a method of growing a human or animal organ or implant comprising: implanting a component cell of the organ on/within the receptacle of the first aspect; implanting further, a different component cell of the organ on/within a further receptacle; stacking the receptacles such that they are in fluid communication; and providing a growth medium to the receptacles to promote growth of the cells.

Any optional or preferable features described in relation to any one aspect of the invention may be applied to any further aspect, wherever practicable.

### DETAILED DESCRIPTION

Practicable embodiments of the disclosure are described below in further detail, by way of example only, with reference to the accompanying drawings, of which:

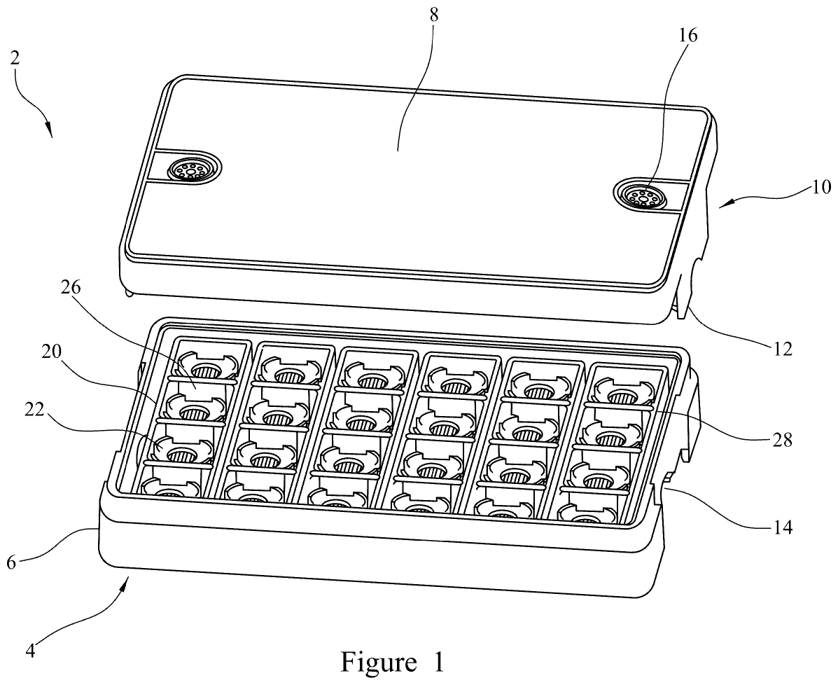

FIG. 1 shows a perspective view of cell culture system;

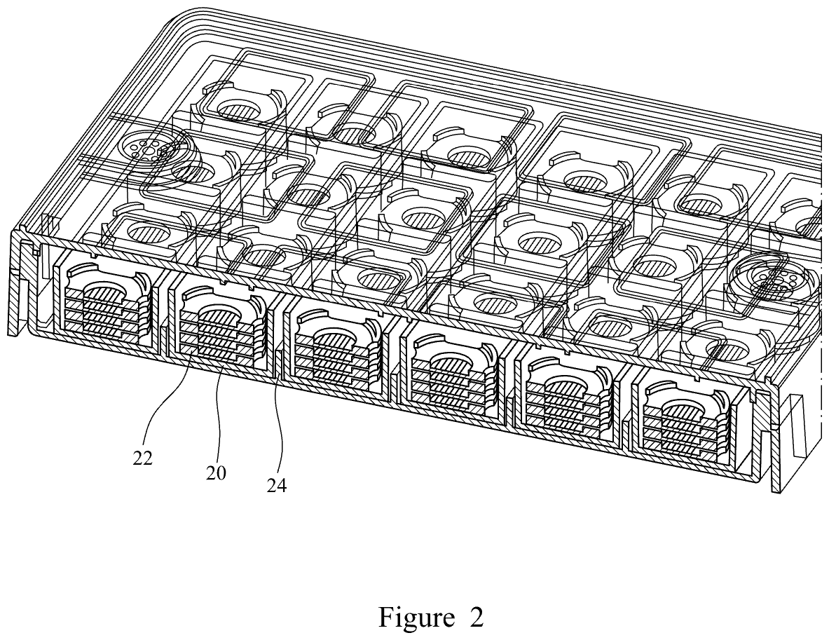

FIG. 2 shows a cross-sectional view of the cell culture system;

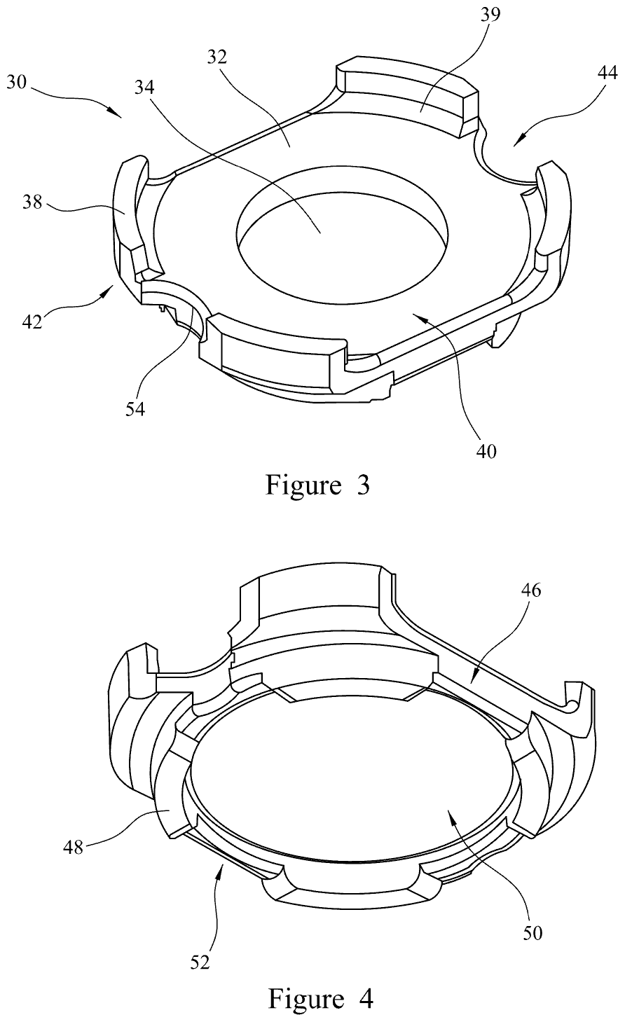

FIG. 3 shows a top perspective view of a cell culture receptacle;

FIG. 4 shows a bottom perspective view of the cell culture receptacle;

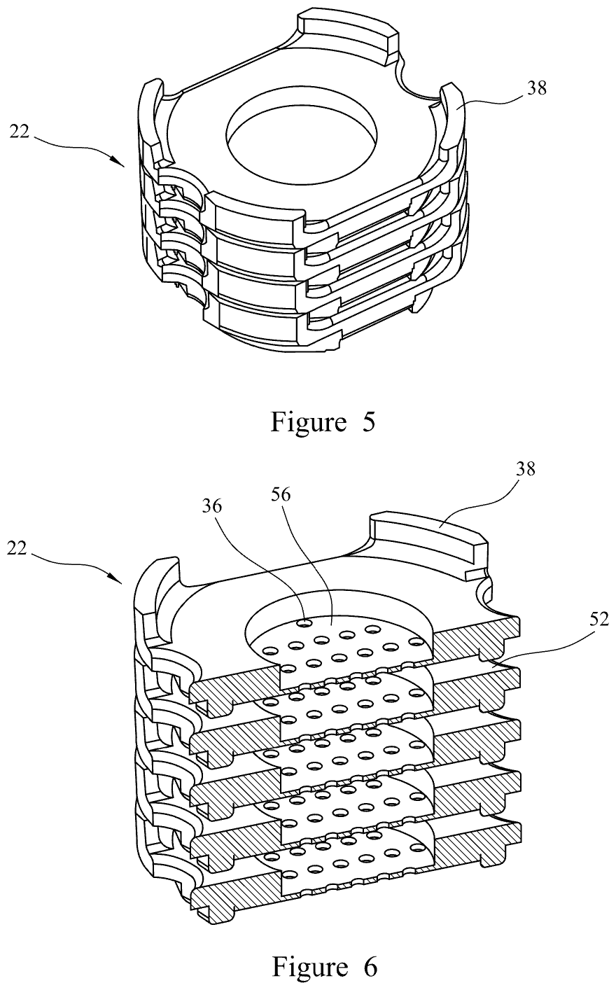

FIG. 5 shows a perspective view of a cell culture block;

FIG. 6 shows a cross-sectional view of the cell culture block;

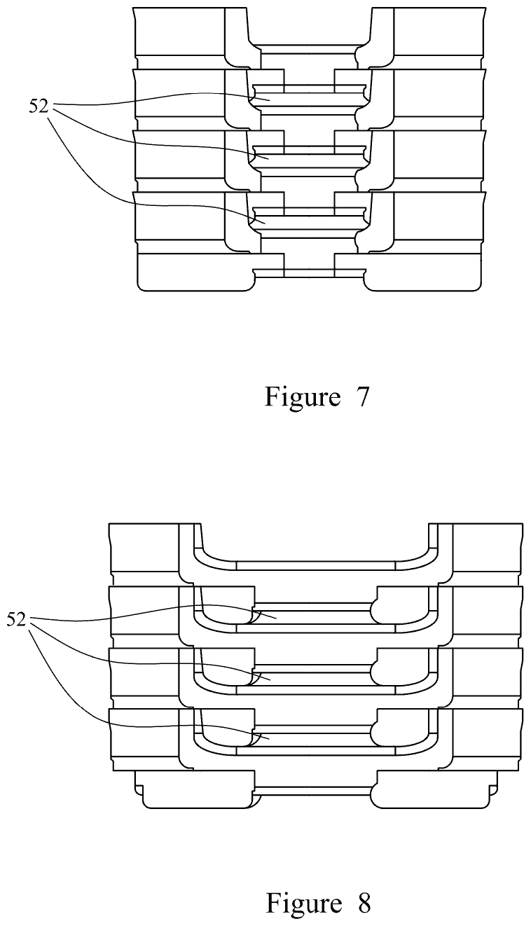

FIG. 7 shows a first side view of the cell culture block;

FIG. 8 shows a second side view of the cell culture block;

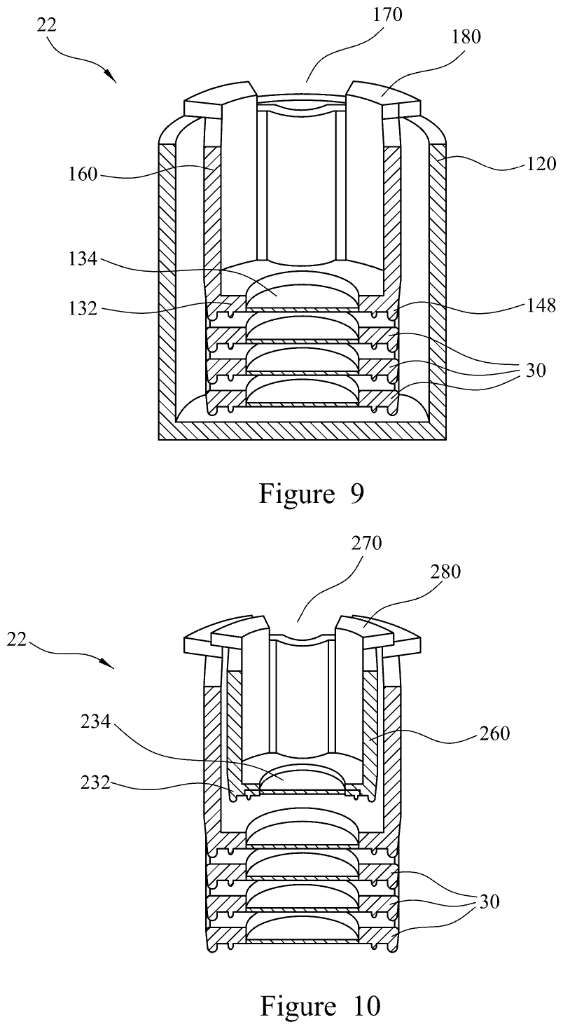

FIG. 9 shows a cross-sectional view of a further embodiment of a cell culture block;

FIG. 10 shows a cross-sectional view of a further embodiment of a cell culture block;

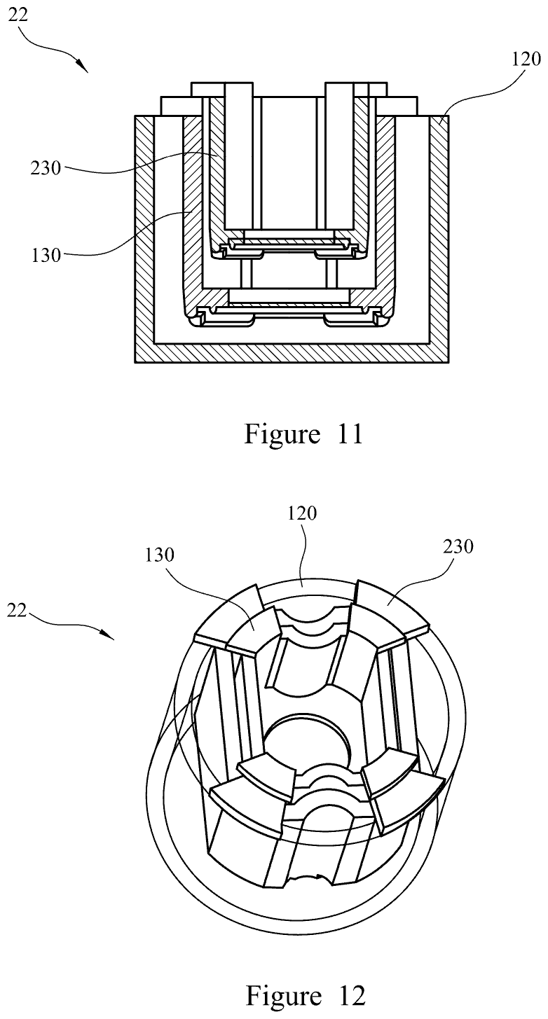

FIG. 11 shows a cross-sectional view of a further embodiment of a cell culture block;

FIG. 12 shows a perspective view of the cell culture block of FIG. 11;

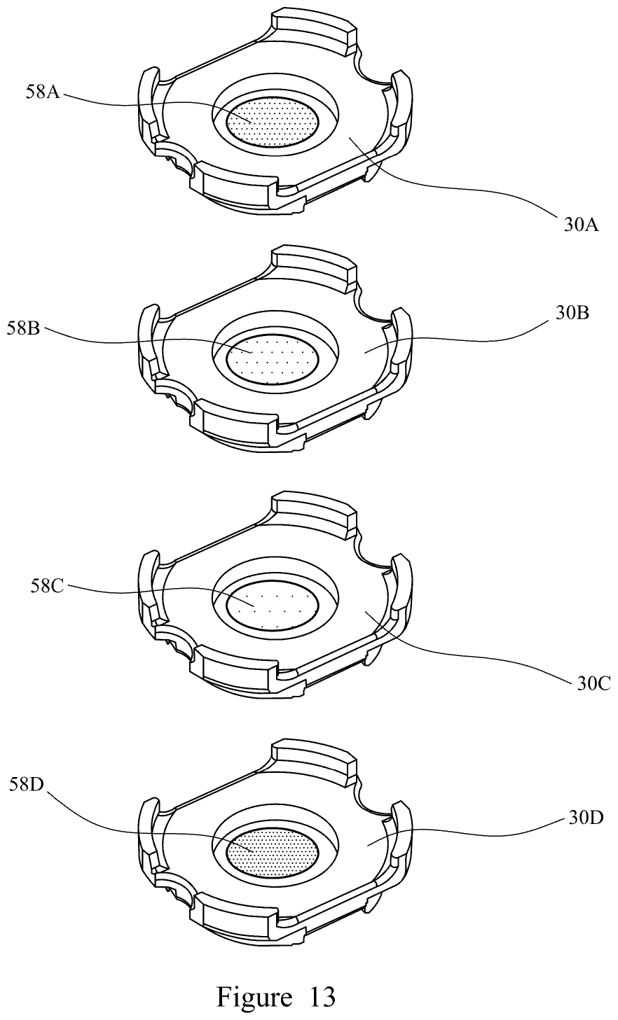

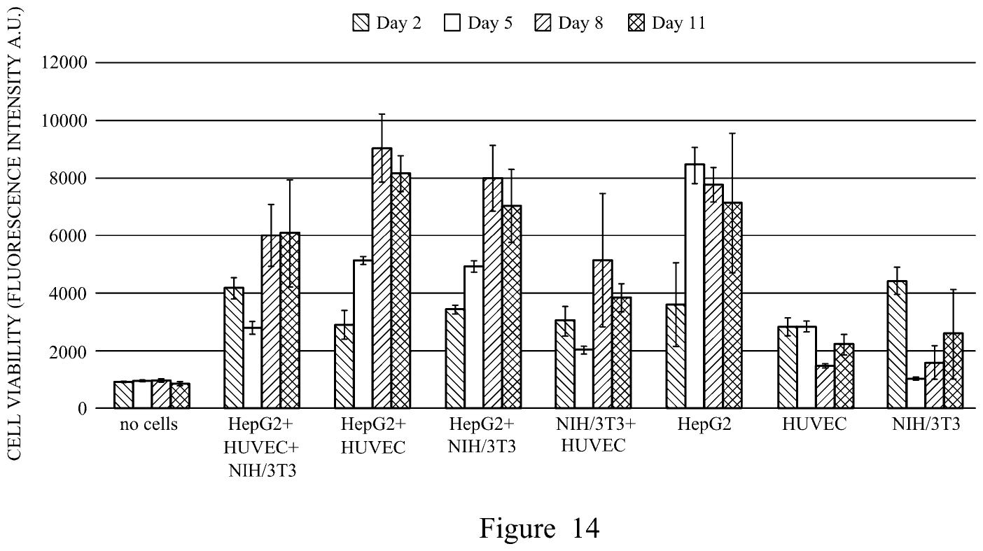

FIG. 13 shows a plurality of receptacles filled with different cell types;

FIG. 14 shows a graph of the viability of a plurality of cell types for the system.

A cell culture system 2 is shown in FIG. 1. The system 2 comprises a tray 4. The tray 4 is configured to provide a housing/container or the like. The tray 4 comprises a base 6. The tray comprises a lid 8. The base 6 and lid 8 attach to form a substantially sealed container. In some embodiments, the lid 8 may be constructed with a loose fit to allow gas exchange.

The base 6 and lid 8 comprises an attachment mechanism 10 to allow releasable attachment thereof. The attachment mechanism 10 comprises a latch 12. The latch 12 is resiliently mounted to the lid 8 and configured to engage a corresponding formation 14 on the base. The latch 12 is resiliently mounted to the lid 8.

The tray 4 may be substantially transparent and/or translucent to allow inspection of its contents without removal of the lid 8. In some embodiments, the tray 4 comprises a window to allow internal inspection thereof. The window may comprise a different material to the remainder of the tray 4. The window may comprise a material suitable for use with a particularly imaging technique/spectroscopy technique (ie transparent to the emitted/probing radiation). The window may comprise a material suitable for Raman spectroscopy (eg transparent to visible, near-IR, or near-UV).

In some embodiments, the tray 4 may be opaque. In some embodiments, the tray 4 may be opaque to one or more select radiation type. For example, the tray 4 may be opaque to UV radiation, but transparent to IR or visible radiation.

The tray 4 is rectangular. The tray 4 is generally planar (ie comprises a shallow depth/height). The tray 4 is sufficiently low in height to fit under a conventional microscope. For example, the container has a height less than or equal to 22 mm. The tray 4 may comprise any suitable dimensions, depending on the specific purpose. The tray 4 may comprise a width of between 25 mm and 250 mm.

The tray 4 may comprise one or more of: polycarbonate; cycloolefin, polyethylene; polystyrene; acrylic copolymer (AV); polyvinyl chloride (PVC); polypropylene (PP); glass (borosilicate); ceramics; metallic materials (eg stainless steel); polycarbonate; cyclo-olefine; Silicones (eg PDMS). Typically, the base 6 and lid 8 comprise the same material.

The tray 4 may comprise one or more air filters 16. The air filters 8 may prevent microbes entering the system. The air filters 16 may be provided adjacent the edge of the lid 8. The air filters 16 may comprise any suitable construction. The air filters 16 may comprise hydrophobic PTFE (Teflon) filters. The air filters 16 may allow gas exchange, but prevent ingress of microbes. The air filter 16 may comprise a pore size less than 1 μm in size; preferably, less than 0.4 μm. The filter 16 may comprise a pore size of 0.2 μm

A plurality of sub-containers 20 are provided within the tray 4. The sub-containers 20 provide a tray or the like configured to support a plurality of cell blocks 22, which will be described in detail later. The sub-containers 20 are configured to be self-contained (ie there is no significant interaction/fluid transfer between respective sub-containers 20). The sub-containers therefore provide dividers or the like configured to divide the tray 4 into a plurality of separate compartments. The sub-containers 20 comprise a tray like form. The sub-containers 20 may be elongate. This provides a line of cell blocks 22. The sub-container 20 may comprise a similar construction and/or material as the aforementioned tray 4.

The sub-containers 20 are removable from the tray 4 to allow individual inspection thereof. The sub-containers 20 may be mounted to the tray 4 via a removable attachment means. The attachment means may comprise one or more: a friction fit; interference fit; fasteners etc. The tray 4 comprises locating features 24 to ensure correct placement of the sub-container 20 within the tray 4 and/or prevent excessive movement thereof. In the present embodiment, the locating features comprise a flange or ridge 24 configured to be received between respective sub-containers. However, it can be appreciated that the locating features can comprise any suitable formations, for example, any suitable protrusion or recess provided on one or both of the tray 4 and the sub-container. For example, the sub-container 20 may comprise a detent configured to be received within an indent on the tray 4. Locating features are provide for each sub-container 20 accordingly. The tray 4 may contain between 2 and 30 sub-containers.

The sub-container 20 typically extends the whole width/length of the tray 4. In other embodiments, the sub-container may extend only a portion of the width/length of the tray 4. Typically the sub-containers have a width dimension configured to contain a single cell block 22. For example, the sub-container comprises width dimension between 15 mm and 10 mm. The sub-container 20 comprises a length configured to received multiple cell blocks. For example, the sub-container 20 comprises length of approximately 80 mm. In some embodiments, the sub-container 20 comprises a N×N grid of cell blocks 22. The sub-container 20 may extend substantially the full height of the tray (eg greater than 90% thereof).

In some embodiments, the sub-containers 20 are stackable. The sub-containers 20 may comprise locating/stacking features. For example, complementary protrusions/recesses may be provided.

The sub-container 20 is divided into multiple compartments by a divider 26. The dividers 26 extend across the width of the sub-container 20. The dividers 26 extend up the majority of or all of the height of the walls of the sub-container 20. In some embodiments, the divider is porous/diffusive. The divider 26 may comprise selective permeability (ie only allow select agents to pass therethrough). The divider 26 may have the same porosity as a biological barrier, for example the blood-brain barrier, and therefore allows the same types of molecules to pass through.

The divider 26 comprises a supporting frame 28 which supports the filter material. The supporting frame 28 comprise a channel or recess configured to receive the divider 26. The supporting frame 28 may extend all or a portion of the interior surface of the sub-container 20. The supporting frame 28 is moulded/integral with the sub-container 20. In some embodiments, a divider 26 is place on top of the whole or portion of the sub-container 20. For example, a divider may be placed on top of or over one or more cell block 22. Supporting frames may be provided at the upper end of the sub-container 20 accordingly. In the present embodiment, three dividers are provided. However, it can be appreciated this is merely exemplary and any number of dividers 26 and/or supporting frames 28 may be provided. The tray 2 may be configured to provide support for between 2 and 400 cell blocks.

Dividers 26 may be placed in the sub-container 20 within the supporting frame using aseptic techniques such as sterilised tweezers or a kit may be provided. The user may place dividers 26 as required. The dividers 26 may comprise a membrane or the like. The membrane may comprise nylon. The membrane may comprise a pore size of 2 μm.

Referring now to FIGS. 3-8, the cell block 22 is described. The cell block 22 comprises a plurality of discrete, individual receptacles 30. Each individual receptacle 30 is configured to culture a cell. The receptacles 30 may be stacked or piled to allows the cell block 22 to culture different cells. This allows culture of a plurality of different cells in a close/adjacent formation to replicate an organ etc. Although four receptacles 30 are shown as forming a cell block 22 in the figures, it can be appreciated the actual number of receptacles is arbitrary and the receptacles 30 can be stacked as high as required.

An individual receptacle 30 is shown in FIGS. 3 and 4. The receptacles 30 provides a substantially planar, plate, puck or disc like form. The plate 30 comprise a body portion 32. The body 32 comprises a generally rigid structure. The body 32 comprises a polymeric material, for example, one or more of: polystyrene (PS); acrylic copolymer (AV); polyvinyl chloride (PVC); polypropolyne (PP); polycarbonate; cyclo-olefine; or silicones (eg PDMS). In some embodiments, the body 32 comprises one or more of: ceramics; metallic materials (eg, stainless steel); or glass (eg borosilicate). The plate 30 may be transparent, semi-transparent or opaque. In some embodiments, the plates 30 may be porous. For example, the plates may comprise a mesh, grid, grille, or textile etc. The plates 30 may comprise a flexible material.

A well 34 for culture of cells is provided within the body 32. The well 34 comprise a recessed portion for containing the cells in use. The wall 34 may comprise the same material as the body 32 and/or be integral therewith. The well 34 comprises a portion of reduced thickness. For example, the well 34 may comprise a thickness of less than 1 mm. The well 34 is circular in the present embodiment. However, the well 34 may comprise any suitable size or shape, as required.

The plates 30 comprises stacking features to aid with stacking of the discs to ensure correct spacing therebetween and/or allow fluid flow between the plates 30. The stacking features also prevent relative movement between the plates 30. This prevents the plates 30 sliding relative to one another (ie in the plane defined by the plate 30). The plates 30 comprise a raised protrusion. In the present embodiment, the protrusions comprise a lip/flange 38. The lip 38 is provided on the upper side 40 thereof. The lip 38 is provided at the corners 42 of the plate 30. The lip 38 is provided as a plurality of discrete/individual portions. Gaps 44 are therefore provided between the lip portions 38. The gaps 44 extend along the sides 46 of the plate 30. The gaps 44 allows fluid to flow into/over the plate 30 in use.

Similar lips/flanges 48 are provided on the underside 50 of the plate 30. The underside lips 48 are provided radially inward relative to topside lips 38. This helps to prevents relative lateral movement. The underside lips 48 thus are received within the topside lips 38 when the plates are stacked on one another. The lips 38,48 may abut and/or face one another when stacked. The topside lips 38 engage/abut the lower end of the adjacent the topside lips 38. The underside lips 48 engage/abut the body 32.

In some embodiments, the lips 38,48 may engage one another to provide resistance therebetween. The plates 30 may therefore connect via a friction/interference fit. In other embodiments, the lips 38,48 may be loosely spaced such that the plates 30 are loosely connected (ie merely rest on one another).

The underside lips 48 are discrete and spaced to provide respective gaps 50. As best seen, the respective gaps 44,50 define gaps 52 between the respective plates 30. The gaps 52 are provided on each side 46 of the plate, thus allowing flow through the plates from any horizontal direction (ie the gaps 52 extends in orthogonal directions). In the present embodiment, fluid may flow in number of different non-parallel directions (eg in perpendicular direction). This allows greater flow and mixing into the system.

The gaps 52 may extend only part way along the height of the lips 48 (ie the gaps 52 comprise a partial lip). The gaps 52 there provide a constriction or narrowed portion. The size of the gaps 52 may be tuned to vary the flow through the system.

In general terms, the gaps 52 form through holes or channels passing through/between the plates. The gaps 52 may define a substantially X-shaped cavity/channel between the plates 30. As such, the space defined between the plates 30 is in fluid communication with the external/surrounding environment. The space between plates 30 is therefore in fluid communication with each of the other spaces between adjacent plates in the same sub-container. The gaps 52 comprise a height (ie distance between adjacent plates 30) of less than or equal to 10 mm, less than or equal to 5 mm, less than or equal to 3 mm, or less than or equal to 1 mm. The gaps 52 comprise a height of greater than or equal to 0.1 mm, 0.3 mm, 0.5 mm or 0.7 mm. The relatively small gaps between adjacent plates may facilitate imaging of the cell layers through the stack of plates 30 using standard inverted cell culture microscope without the need to remove individual plates 30.

The topside lips 38 may comprise a ramped/curved/tapered interface between the lips 38 and the body 32 of the receptacle. This helps locate/guide the underside lips 48 into the correct position. This also provides a space between the respective faces topside lips 38 and the underside lips 48. Fluid may therefore flow between the lips 38,48 (ie in a circumferential direction).

In some embodiments, the topside lips 38 and/or the underside lips 48 may be substantially continuous. The gaps 52 may therefore be provided by apertures or other permeable/porous members therein. For example, the gaps 52 may comprise a selective membrane.

The lips 38,48 are curved/arcuate/rounded. The lips 38,48 may collectively define a circular or ovate shape. The lips 38,48 are provided about a perimeter/periphery of the plate 30 (ie a side thereof). The protrusions 38 generally comprise any suitable form. The protrusions may comprise any suitable raised ridge, detent, rim or projection. In some embodiments, the plate 30 may comprise a recess to receive a corresponding protrusion. The recess/protrusion may provide an interlocking formation. The recess may comprise any suitable groove, indent, channel etc. For example, a recess may be provided in the upper side 40 on the plate 30 and configured to receive the underside lip 48. The recess may generally only receive a portion of the protrusion to ensure a gap is defined between the plates 30.

The lips 38,48 may comprise a variable size. This may allow variable spacing between different plate 30. A kit of parts may be provided comprising plates 30 with varying size lips 38,48.

The sub-container 20 may comprise stacking/locating formations configured to engage the cell blocks 22. This helps to locate the blocks 22 and/or prevent undesired movement thereof. The sub-container 20 may be shaped or sized to receive the underside 50 of the plate 30. The sub-container 20 may comprise a recess to receive the underside lips 48. Additionally or alternatively, the sub-container 20 may comprise a protrusion to engage the lower end of the topside lips 38. It can be appreciated that each sub-container 20 comprise locating features for each of the cell blocks 22 location therein. The sub-container 20 may be configured to contain between 2 and 20 cell blocks.

In some embodiments, the tray 4 comprises locating/stacking features to receive the cell block 22, as previously described. Thus, in some embodiments, some or all of the cells blocks 22 are received directly in the tray 4.

The plates 30 comprises a groove/indent 54 at a side 46 thereof. The indent 54 extends radially inward. The indent 54 allows tools (eg tweezers) to be inserted between adjacent block 22 to allow retrieval thereof. A pair of indents 54 on opposing sides 46 of the plate 30 are provided in the present embodiment. However, it can be appreciated indents may be provided on any or all sides 46 of the plate 30. In some embodiments, the sub-container 20 comprises protrusion configured to be received within the indents 54 to provide location thereof.

In some embodiments, the tray 4 and/or sub-container 20 comprises inlets and/or outlet to allow fluid flow in and/or out thereof. For example, the inlets/outlets may provide flow of a growth medium.

The well 34 may be porous/diffusive. This allows cells, growth medium and/or other fluids to pass through/around the plates 30. As best seen in FIG. 6, this provides an effective cell growth cavity defined between the plates 30. The well 34 may be perforated. The well 34 may comprise apertures 36 therein. The apertures 36 may extend through the plate 30. The size and/or number of apertures 36 may be determined according to the desired flow rate through the plates. The well 34 may comprise a mesh, grid, grille, or textile.

In some embodiments, the porosity is provided by a filter/membrane. The filter/membrane may be selective. The filter may be hydrophilic or hydrophobic. The filter may allow nanofiltration, microfiltration or ultrafiltration of substances between cell growth blocks. The pore size of the filter may range from 1-100 nm, for example: 1 -20 nm; 20 -40 nm; 40 -60 nm; 60 -80 nm; 80 -100 nm. The pore size of the filter may range from 1 -100 μm, for example: 1 -20 μm; 20 -40 μm; 40 -60 μm; 60 -80 μm; or 80 -100 μm. In some embodiments, the pore size is greater than 100 μm

The filter may comprise a range of organic and/or inorganic materials including one or more of: metals; polymers (natural and synthetic); glass; or ceramic. The filter may comprise one or more of: cellulose based filters (eg cellulose acetate, cellulose nitrate, mixed cellulose esters); polyether sulfone (PES); PTFE (polytetrafluoroethylene); silica gel membrane; Nylon; polyester; polycarbonate; polyester (PET) membranes; metallic material (eg steel); ceramic material; and/or combinations thereof. The filter may comprise a single layer. Alternatively the filter comprises multiple layers. The respective layers may comprise one or more of the above materials.

By modifying the thickness, pore size, or composition of the filter different in vivo barriers can be simulated. For example, the filter may emulate the blood brain barrier or kidney glomerulus. The delivery of test agents can be controlled to replicate in vivo conditions, for example, for toxicity testing.

The filters may be used for protein extraction, purification of virus filtration, buffer exchange, sterilisation, blood plasma fractioning, DNA extraction or collection of biological material (DNA, protein etc) released by cells which is achieved by using filters with different pore size. For example, a large pore ultrafiltration could be constructed to filter molecules of sizes ranging from 100-500 kDa. For instance, virus filters and low protein binding filters might be composed of polyvinylidene fluoride (PVDF), polyether sulfone (PES), or cuprammonium regenerated cellulose filters with pore size ratings of 15 nm-75 nm.

In some embodiments, the well 34 may be solid/non-porous. The respective wells 34 are therefore fluidly connected by the gaps in between in the plates 30.

The well 34 contains a cell growth area. The cell growth area may comprise a scaffold 56. The scaffold 56 may be incorporated into structure of the plate 30 (eg physically or chemically). The scaffold 56 may be retained by the supporting mesh of the supporting frame. The scaffold 56 may be retained on the cell growth area which might be porous or non-porous. The filter might be coated with plasma surface charge to improve cell adhesion or the attachment of a scaffold.

The scaffold 56 may be added on top the cell growth area such as filter membrane or non-porous cell growth surface. The cell growth area may be coated with plasma before the application of the scaffold. The cell growth area may be coated with plasma to improve its adhesion to cells or proteins or scaffold. The cell growth area may be compromised of a scaffold to support the growth of cell cultures (2D and 3D).

The scaffold may be composed of natural or synthetic polymer, or hybrids of natural and synthetic polymers to create three-dimensional in vitro microenvironments to mimic the extracellular matrix (ECM) of native cells and tissues. The scaffold material may be any suitable material, for example: polymers; (including hydrogels); tissue constructs; metals; glasses; or ceramics. The scaffolds may be formed of synthetic or natural substances. The scaffolds, either individually or in combination, are incorporated into cell growth blocks to mimic the extracellular matrix (ECM) of natural living cells in laboratory conditions. Preferably, the scaffold is a polymer scaffold. Most preferably the scaffold is a hydrogel scaffold. By hydrogel is meant a polymeric gel in which the liquid component is water.

The scaffolds may comprise one or more natural polymers, for example proteins (such as collagen, fibrin, alginate, gelatine, silk and/or genetically engineered proteins), polysaccharides (such as agarose, carboxymethylcellulose, hyaluronic acid and/or chitosan), DNA, live cells and tissue constructs or any combination of the above. The scaffolds may comprise one or more biodegradable polymers including polyester containing macromers such as poly (-caprolactone) (PCL), poly glycolic acid (PGA), and poly lactic acid (PLA).

The scaffolds may comprise non-biodegradable polymers, for example polymers of acrylamide (AAm), monoacrylate (mPEGMA or PEGMA), acrylic acid (AAC) and methoxyl poly (ethylene glycol) (PEG) monoacrylate) (mPEGMA), hydroxyethyl methacrylate (HEMA), 2-hydroxypropyl methacrylate (HPMA), acrylic acid (AAc) and N-isopropylacrylamide (NIPAm), or polystyrene (PS). The physiochemistry of the scaffold is selected to be suitable for cell growth.

The mechanical strength of the scaffold can be controlled to maintain the properties of the scaffold such as its strength, longevity, stiffness, roughness, viscoelasticity and/or porosity.

The scaffold stiffness may be modified to replicate differing in vivo cell locations. Different stiffness or roughness is required to culture different cell types (for example a soft scaffold may be required for lung cells, but a hard scaffold may be required for bone cells). Techniques to make hydrogels with specific shear moduli (measured in kPa) and/or specific pore sizes are known (and published). For example, polyacrylamide stiffness is controlled by the relative concentration of acrylamide monomer and its cross-linker. Different types of tissue have different relative stiffness, and the scaffold blocks may be engineered to have a stiffness to match. Liver, mammary, brain, bone marrow and lung cells generally have a stiffness in the range 100 Pa to 2 kPa. A suitable polyacrylamide hydrogel may be produced using 3% acrylamide (v/v) and 0.06% bis-acrylamide (v/v), to give a hydrogel with Young's shear modulus of around 500 Pa. Skin, spleen and kidney cells generally have a stiffness in the range 3 kPa to 8 kPa. A suitable polyacrylamide hydrogel may be produced using 5% acrylamide (v/v) and 0.15% bis-acrylamide (v/v), to give a hydrogel with Young's shear modulus of around 5 kPa.

Cardiac, myoblast, arterial, muscle and skeletal cells generally have a stiffness in the range 10 kPa to 20 kPa. A suitable polyacrylamide hydrogel may be produced using 10% acrylamide (v/v) and 0.1% bis-acrylamide (v/v), to give a hydrogel with Young's shear modulus of around 10 kPa.

Pre-calcified bone cells generally have a stiffness in the range 25 kPa to 40 kPa. A suitable polyacrylamide hydrogel may be produced using 10% acrylamide (v/v) and 0.3% bis-acrylamide (v/v), to give a hydrogel with Young's shear modulus of around 35 kPa.

It will be clear to those skilled in the art that hydrogels of other stiffness may be produced by suitably varying the ingredients. The mechanical properties of the scaffold may be selected to allow shape formation or maintenance of the cells during cell growth. The mechanical properties of a scaffold may be controlled by using a combination of materials. For example, the properties of a hydrogel scaffold may be controlled by using an inorganic or organic material in combination with the hydrogel, for example ceramics, metals, other hydrogels or any other suitable structure.

The scaffold may be porous or non-porous. If the scaffold is porous, the pore distribution, pore structure and pore size can be controlled. If the scaffold is porous, the pores may have a specific pore size (nm, μ|η or mm) to allow the movement of cells, cell signalling molecules, nutrients or test substances either through the matrix, surface or at specified locations or to remove waste substances.

Thus, the scaffold may be selectively permeable to control the diffusion of specific test substances, cell signalling molecules, nutrients and other test agents, for example nanoparticles, viruses, or bacteria, through the scaffold and/or cell growth block, or from one block to another.

The porosity of the scaffold can be modified such that the diffusion of certain substances and their rate of diffusion can be selected. This could influence cell division, cell growth, cell death and other phenotypic changes designated as physiological, pharmacological or toxicological.

The scaffold material may allow the controlled diffusion, through its modified porosity, of agents (pharmaceuticals, toxins, agents, physical entities including nanoparticles, cell factors) towards a cell population growing on the outer surface of the scaffold. This replicates more faithfully exposure settings that occur in vivo within an in vitro setting-situations this could replicate include the diffusion of inhaled entities through lung cells into the systemic circulation, diffusion across the blood brain barrier, diffusion across the GI tract, diffusion across blood vessels such as capillaries into surrounding cells, and diffusion across all barriers (physiological and non-physiological) to influence adjacent cell populations. The scaffold may permit diffusion of substances such as nutrients or drugs.

The scaffold may be manufactured with a pore size that provides a desired diffusion rate for a particular substance, or particular substances. Alternatively, the scaffold may be manufactured such that diffusion rates for the relevant substance(s) are known. For many diffusive materials, reference tables of a wide range of cation diffusion rates and anion diffusion rates are available. Alternatively, diffusion rates may be determined using known diffusion rate test equipment.

The scaffold may have varied pore sizes selected to enhance the growth of different specific cell types. For example, for fibroblasts and epithelial cells, pore size might range from 5 μm to 100 μm, for endothelial cells about 25 μm, and for vascular smooth muscle cells, 63-100 μm. For tissue regeneration a minimum pore size of 100 μm can simulate mitigation conditions but the pore size is preferably 300 μm to improve bone formation and develop a network of capillaries. Degradation of the scaffold can be controlled for particular cell growth, for example for implants used in tissue regeneration.

The surface area of the scaffold may be modified and/or activated by incorporation of active functional groups (for example peptides) to increase support for cell growth and for mitigating scaffold degradation. The polarity of the scaffold may be modified to increase cell adhesion and the spreading of living cells, for example by adding proteins, changing the charge of the surface groups, or by the addition of peptides. Cell adhesion may be increased by inserting structural motifs within the scaffold. For example, a negative surface charge can increase cell attachment.

The scaffold may be supported by other materials to give a specific shape (eg square) or to maintain its structure. The supporting material for the scaffold block may be any suitable material, for example, plastic, metal, ceramic or glass. Antibodies, scaffolds or other structures (including nanostructures) could be adsorbed onto the surface of these supporting materials to selectively adhere or manipulate specific cell types for selective clonal expansion or isolation from a particular cell population. Other proteins such as plasma proteins (eg albumin) may be contained within a scaffold; these are commonly found in blood and influence the kinetics (absorption, metabolism and excretion) of pharmaceuticals and toxic agents. The scaffold may be formed with an outer layer which may be advantageously modified to improve cell attachment and better support cell growth and differentiation. Such a layer may comprise hydrogel. In particular, a layer of hydrogel may include an outer surface coated with an Extra-Cellular Matrix (“ECM”). A hydrogel outer layer of the scaffold may be formed to match one or more properties of the cells and/or tissue desired to grow there. Such properties may include rigidity, stiffness and/or other native properties of different body tissues. The outer layer may constitute the housing for the cell growth block as discussed above.

A combination of scaffold materials may be provided to create a matrix suitable for cell growth. A plurality of different surfaces may be provided in different regions of the device to encourage growth of different types of cell.

The surface of the scaffold may be provided with microscopic roughness and/or macroscopic structure to provide favourable growth environments for specific types of cell. The scaffold material may allow the controlled diffusion, through its modified porosity, of agents (pharmaceuticals, toxins, agents, physical entities including nanoparticles, cell factors) towards a cell population growing on the outer surface of the scaffold. This replicates more faithfully exposure settings that occur in vivo within an in vitro setting-situations this could replicate include the diffusion of inhaled entities through lung cells into the systemic circulation, diffusion across the blood brain barrier, diffusion across the GI tract, diffusion across blood vessels such as capillaries into surrounding cells, and diffusion across all barriers (physiological and non-physiological) to influence adjacent cell populations in other cell growth blocks.

The system 2 may be compartmentalised with scaffold blocks to allow for co-culture of differing cells types with or without the different cell types coming into contact, (eg fibroblasts and epithelial cells, differing epithelial cells, bacteria/virus particles and mammalian cells). Designated filtration pores may allow transfer of factors from one cell type to another. This enables the study of the effects of one cell on another. Co-culture systems are composed of at least two different cell types in order to simulate cell-cell interaction of the in vivo microenvironment of natural tissue, for example in cancer studies. Experimental conditions that the device may replicate include breast cancer models, by co-culturing breast carcinoma or parenchymal cells with stromal cells (ie fibroblasts adipocytes, lymphocytes and epithelial cells), human skin models by co-culturing dermal fibroblasts with keratinocytes, and healthy or damaged neurones, by co-culturing peripheral and central nervous systems in combination of glia cells (eg, astrocytes) together with oligodendrocytes.

Different eukaryotic cells (eg fibroblasts, epithelial cells), prokaryotic cells with eukaryotic cells, viral particles with eukaryotic cells or viral particles with prokaryotic cells may be studied. The scaffold prevents actual cell-to-cell contact but permits the diffusion of factors from one cell type to another in order to ascertain their effect on the other cell type. The true effects of stromal factors or infectious secretions in isolation of cell-to-cell contact can be examined. Scaffold blocks may be provided with or without cells, and may require modification prior to use.

A cell block 22 may contain cell growth receptacles 30 and another compartment separated by a divider 26 may contain media incorporating a test substance (eg drugs, chemical pollutants, viruses, bacteria) and the diffusion of the test substance can then be controlled to replicate living conditions. The cell block 22 will serve as a diffusive layer to control the movement or selection of material allowed to cross the barrier. For example, cancerous cell culture can be exposed to the testing substance at a diffusion rate that is controlled to replicate the environment of living tissue. Multiple dividers 26 may be used to separate the container into a desired number of compartments. The dividers 26 may be layered or stacked. Alternatively, the cell block 22 and the test substance may be separated by multiple compartments.

The cell growth blocks 22 may all be located in one compartment. In some embodiments, cell growth blocks 22 may be located on one side of a divider 26 and further cell growth blocks 22 may be located on the other side thereof. This enables the user to have control over which cell growth blocks are exposed to which test substances.

The dividers 26 may be made of the same material as the scaffolds described above. This may include one or more of: natural and/or artificial polymers (including hydrogels); tissue constructs; metals; glasses and ceramics.

The cell blocks 22 may be used for co-culture of different cell types of human cells or mammalian cells such as fibroblasts, endothelial cells, epithelial cells immune cells. The cell blocks 22 may be used to simulate different organs for example skin layers, liver layers, blood-brain barrier, GI tract, kidney tissue. Skin barriers may also be created using artificially composed polymers. Skin barrier may be incorporated by including keratinocytes, Melanocytes, Langerhans cells.

The blood brain barrier may be simulated by using a co-culture of cells types such as epithelial cells, astrocytes, or pericytes.

Liver may be simulated using hepatocytes, liver endothelial sinusoidal cells (LSCs), Kupffer cells, stellate cells. Liver may be composed of primary or no primary cells or combination of primary and nor primary cells. For example, Primary hepatocytes may be co-cultures in nanoblock layers with mouse fibroblasts such as NIH/3T3.

Sensors can be incorporated in all of any one of the cell block 22, sub-container 20 and/or tray 4. The sensor(s) may measure inflow or outflow of media and gas content. The sensors may be removable. The sensors may comprise one or more of: analytical; spectrochemical; electrical; elemental; or optical sensors. The sensor may comprise nanosensors (eg based on graphene, C60 fullerene, or other nanoparticles). The sensors can be used to sense electrical/chemical impulses or movements of differing cell types; track the development of either single cells or populations of cells; determine cell-to-cell interactions; respiration of cells; varying hardness and thickness of individual cells or cell colonies; determine cell lineage (the hierarchy of cells within a given cell population from stem cell to differentiated cell); and/or cell-to-cell signalling (eg via chemical factors, enzymes, reductive or oxidative processes, exosomes, proteins, liposomes, RNA or DNA molecules, viral or nanoparticles)

Cells may be grown on upper side 40 and/or the underside 50 of the receptacle 30. Cells may therefore communicate and/or be spaced via the porous well 34.

In use, cells can be implanted within the scaffold at varying depths in order to modify oxygen tension (ie the partial pressure of oxygen present). This allows replication of the varying oxygen tension one might find in a human tissue or disease state. For instance, as one enters the crypt of the GI tract the cells hidden at the base of the crypt are likely protected from the damaging effects of high oxygen tension and are likely to contain stem cells. In cancer, as a tumour grows there are hypoxic regions—these may contain cancer stem cells and are less amenable to treatment. This device allows one to replicate these scenarios. Colonies of cells can be implanted into the scaffold.

The device 2 may be used to produce surgical implants. The device may be pre-incubated in order to populate the implantable material with specific cell types (eg osteocytes) prior to surgical implantation. This implantable material could be populated with stem cells in order to allow cell adhesion of donor cells for autotransplant, allotransplant or xenotransplant.

Spaces could also be created in the device for input of removable implant material. This would allow transfer-culture of cells on the scaffold to the implant prior to use of an implant in surgery. This could allow for growth of bone, cartilage or arteries for example. The device may also be used as an in vitro variable simulation of the in vivo basement membrane. For instance, one scaffold's stiffness can replicate the prostate epithelial cell basement membrane whilst another can replicate that in breast tissue. A basement membrane is a modifiable substrate upon which differing cell types can grow and differentiate. The roughness of the scaffold may determine the ability of specific cell types to adhere to the scaffold.

In order to use the system 2, a scaffold or other cell growth structure is provided on the cell plate 30. One or more cells are implanted onto the plates 30. The plates 30 are then assembled/stacked according to the desired characteristics. The plates 30 are then placed into the sub-container 20 as a cell block unit 22 or individually. The sub-container 20 is then placed into the tray. Growth medium may then be placed into the sub-container 20 and/or tray 4 as required. It can be appreciated that such steps may be performed in any suitable order, for example, the scaffold and/or cells can be implant whilst the plates 30 is located within the sub-container 20 and/or tray 4.

Referring now to FIGS. 9 to 12, the cell block unit 22 may comprise components in addition to the plates 30 discussed above. In particular, the uppermost component of the cell block unit 22 may be a primary barrier plate 130, which may comprise a body portion 132, cell growth area 134 and lips/flanges 148 on the underside for engagement with the lip/flange 38 on the upper side of a plate 30 immediately below. However, instead of the lip/flange 38 of the plates 30, the primary barrier plate 130 comprises a barrier 160 that is configured such that the cells in the cell growth area 134 are in communication with plates 30 only through the cell growth area 134. Accordingly, the cell growth area 134 of the primary barrier plate 130 may be selectively permeable. The primary barrier plate 130 may further comprise an open top 170 and projections 180 that may engage with the rim of a sub-container 120, which may be a well of a standard multi-well plate.

The cell block unit 22 may further comprise a secondary barrier plate 230 comprising a body portion 232, cell growth area 234, barrier 260, open top 270 and projections 280. The secondary barrier plate 230 may be inserted into the open top 170 of the primary barrier plate 130 and the projections 280 may engage with a portion of the primary barrier plate 130, such as the projections 180. It is conceivable that the cell block unit 22 could comprise additional barrier plates, for example a tertiary barrier plate (not shown) insertable within the open top 270 of the secondary barrier plate 230.

In configurations in which a secondary barrier plate 230 is inserted into the open top 170 of a primary barrier plate 130, there may be an opening sufficient to permit gas flow between the primary barrier plate 130 and the secondary barrier plate 230, such as between the inner surface of the first barrier 160 and the outer surface of the second barrier 260.

As depicted in FIG. 11, it is possible for cell block unit 22 to comprise only barrier plates, such as a primary barrier plate 130 and a secondary barrier plate 230.

### Specific Example

In a specific example, the system is used to emulate a human liver. With reference to FIG. 13, immune cells 58A are placed on an extra-cellular matrix provided the well 34 of a first receptacle 30A; similarly, endothelial cells 58B in a second receptacle 30B; stellate cells 58C in a third receptacle 30C; and hepatocytes 58D in a fourth receptacle 30D. The receptacles 30 are porous, thus allowing fluid communication therebetween. The receptacles 30 are stacked to form a cell block 22. Further cells blocks 22 are produced accordingly, and placed into a tray 6 into 6x4 array of blocks 22. Growth medium is supplied and the cells are grown as is conventional.

FIG. 14 shows the viability of various liver cells using the present system. It can be seen that the various cells have viability for times in excess of 11 days.

The present system provides a plurality of fluidly connected receptacles for growth mediums. This allows a plurality of different cell types to be placed therein, thus more closely emulating in vivo conditions for those cell types due to the increased interaction therein. This allows better investigation and/or production of various cell types.

The modular and stacking cell blocks allow a flexible configuration that is easy to handles and assemble, to provide a flexible configuration. The size of the plates and the gaps there between are optimised to ensure adequate flow through/between plates.

1. A cell culture system comprising:
a receptacle having a cell growth area;
a stacking formation comprising a recess or protrusion configured to abut an adjacent receptacle in use to limit relative movement therebetween in at least one direction;
the stacking formation configured to space adjacent receptacles and comprising at least one gap therein, such that the space defined between the receptacle is in fluid communication with an environment external to the receptacle.

2. A cell culture system according to claim 1, where the stacking formation comprises a first protrusion on a first side of the receptacle and a second protrusion on a second side of the receptacle.

3. A cell culture system according to claim 2, where the first protrusion is configured to be received within the second protrusion when stacked with an adjacent receptacle.

4. A cell culture system according to claim 3, where the second protrusion comprise a ramped or curved portion configured to space opposing faces of the first and second protrusions to allow fluid to flow therebetween in use.

5. A cell culture system according to any of claims 2 to 4, where the both the first protrusion and the second protrusion comprise gaps therein.

6. A cell culture system according to any preceding claim, where the stacking formation comprises a plurality of gaps, such that fluid may flow between the receptacles in two non-parallel directions.

7. A cell culture system according to any preceding claim, where the stacking formation comprises one or more raised lips or flanges adjacent a side of the receptacle.

8. A cell culture system according to any preceding claim, where the gap comprises a raised protrusion, the protrusion extending away from the receptacle to a lesser extent than the stacking formation such that gap the comprises a constriction or narrowing.

9. A cell culture system according to any preceding claim, where the stacking formation collectively defines a circular or arcuate shape.

10. A cell culture system according to any preceding claim, where the stacking formation provides loose connection of the receptacles.

11. A cell culture system according to any preceding claim, where the stacking formation prevents relative movement of adjacent receptacles in two or more directions.

12. A cell culture system according to any preceding claim, where the spacing between the receptacles at the gap is less than or equal to 1 mm.

13. A cell culture system according to any preceding claim, where the receptacle comprises an indent or recess on one or more side to allow handling thereof by a tool in use.

14. A cell culture system according to any preceding claim, where the receptacle is received within a container configured to fluidly isolate the receptacle from the environment or a further receptacle.

15. A cell culture system according to claim 14, where the container comprises a porous divider configured to separate at least two of the receptacles.

16. A cell culture system according to claim 15, where the porous divider is removably received within the container.

17. A cell culture system according to claim 16 wherein the container comprises locating formations configured to engage the divider to prevent relative movement therebetween in at least one direction.

18. A cell culture system according to any of claims 14 to 17, where the container or divider comprises a locating or stacking formation configured to engage the receptacle to prevent relative movement therebetween in at least one direction.

19. A cell culture system according to any of claims 14 to 18, where the container comprises one or more air filters.

20. A cell culture system according to any of claims 14 to 19, comprising a further container configured to receive a plurality of the containers, the further container comprising a locating or stacking formation to prevent relative movement between the container and the further container in at least one direction.

21. A cell culture system according to any preceding claim, where the cell growth area is selectively permeable or allows fluid to flow though the receptacle, such that adjacent receptacles are fluidly connected via the cell growth area in use.

22. A cell culture system according to claim 21, where the cell growth area is perforated or porous.

23. A cell culture system according to any preceding claim, where the cell growth area comprises a recess or well formed in a body of the receptacle.

24. A cell culture system according to any preceding claim, where the cell growth area comprises a scaffold or extra-cellular matrix.

25. A cell culture system according to any preceding claim, where a plurality of receptacles are stacked to form one or more stacks or cell blocks.

26. A cell culture system according to claim 25, wherein each stack or cell block comprises from 2 to 20 receptacles, from 2 to 10 receptacles, from 2 to 6 receptacles, or 4 receptacles.

27. A cell culture system according to claim 25 or 26, wherein the receptacles allow fluid to flow therethrough such that the adjacent receptacles are in direct fluid communication.

28. A cell culture system according to claim 25 or 26, wherein one or more receptacles comprise a selectively permeable cell growth area and a barrier configured such that the cells in the receptacle are in communication with the adjacent receptacle only through the selectively permeable cell growth area.

29. A cell culture system according to claim 28, wherein the stack or cell block comprises two receptacles that comprise a barrier adjacent to one another.

30. A method of cell culture comprising:
implanting cells on the receptacle of any preceding claim;
implanting further cells on a further receptacle;
stacking the receptacles such that they are in fluid communication; and
providing a growth medium to the receptacles to promote growth of the cells.

31. The method according to claim 30 further comprising implanting further cells on three, four, five, six, seven, eight or more receptacles and stacking those receptacles such that they are in fluid communication.

32. The method according to claim 30 or 31 further comprising removing, adding or replacing one or more receptacles from the stack and/or rearranging the receptacles in the stack.

33. A method according to any of claims 30 to 32, where the cells comprise cells of a different types, such as cells selected from the component cells of one or more of a liver, kidney, skin, heart, mammary tissue, brain, bone marrow, lung.

34. A method according to any of claims 30 to 33, wherein the cell types are selected such that the stack of receptacles replicates the structure of an organ or biological system.
